(19) United States
(12) Patent Application Publication
(10) Pub. No.: US 2022/0081706 A1
(43) Pub. Date: Mar. 17, 2022

(54) METHOD FOR PREPARING A BIOLOGICAL SAMPLE AND FOR DETECTING BIOLOGICAL SPECIES PRESENT IN THE BIOLOGICAL SAMPLE

(71) Applicant: Commissariat à l'Energie Atomique et aux Energies Alternatives, Paris (FR)

(72) Inventors: Myriam-Laure CUBIZOLLES, Grenoble Cedex 09 (FR); Anne-Gaëlle BOURDAT, Grenoble Cedex 09 (FR); Maud SAVONNET, Grenoble Cedex 09 (FR)

(73) Assignee: Commissariat à l'Energie Atomique et aux Energies Alternatives, Paris (FR)

(21) Appl. No.: 17/447,348

(22) Filed: Sep. 10, 2021

(30) Foreign Application Priority Data
Sep. 11, 2020 (FR) 20 09214

(51) Int. Cl. C12Q 1/6806; G01N 1/40; G01N 33/53

(52) U.S. Cl. CPC C12Q 1/6806; G01N 1/4077; G01N 2021/7786; G01N 2001/4088; G01N 33/5306

(57) ABSTRACT

A method for detecting target biological species in a biological sample, the method being implemented in a device that makes it possible to concatenate the preparation of a sample for a detection via selective capture and a detection via biomolecular amplification.

### TECHNICAL FIELD OF THE INVENTION

The present invention relates to a method for preparing a biological sample and for detecting biological species present in this biological sample.

### PRIOR ART

Patent application EP3222989A1 describes a device allowing biological species present in a sample to be lysed, notably in order to extract DNA molecules with a view to detection via PCR amplification. In contrast, this patent application is limited to detection via amplification of biomolecular type after a lysis of the biological species.

Even though preparation of a sample for immunological detection and preparation of a sample for DNA extraction are often very different, patent application EP3629000A1 for its part presents a preparing method that allows, with a single device and from one and the same sample, a first sample to be prepared with a view to a recognition of immunological type and a second sample to be prepared with a view to an amplification reaction.

However, the latter solution only allows two samples to be prepared and requires the first sample to be removed from the device before carrying out the recognition of immunological type and the second sample to be removed from the device before carrying out the detection via amplification.

For its part, patent application US2018/163270A1 describes the so-called immuno-PCR method. This method is supposed to combine the advantages of a method of immunological type, such as ELISA (ELISA standing for enzyme-linked immunosorbent assay), and those of the PCR method (PCR standing for polymerase chain reaction). This method, which appeared in 1992, allows detection sensitivity to be increased with respect to the conventional ELISA method. Conventionally, it is based on the use of a reagent obtained by coupling an antibody, which is intended to recognize the target, to an oligonucleotide sequence.

The aim of the invention is to provide a method suitable for preparing samples, advantageously without a step of lysing the biological species present in the sample, with a view to a recognition of immunological type (selective capture) and to a detection via biomolecular amplification, this method being fast, reliable, not fractionating the sample and engaging all the targets present in the sample.

### SUMMARY OF THE INVENTION

This aim is achieved via a method for detecting target biological species in a biological sample, said method being implemented in a detecting system that comprises a preparing device and detecting means, said preparing device comprising:
a casing comprising at least one aperture,
a first channel produced in said casing,
a second channel produced in said casing,
a chamber into which emerge the first channel and the second channel,
a filter separating said chamber into two separate spaces so as to define a first space into which emerges said injection first channel and a second space into which emerges said second channel, said filter having a porosity suitable for retaining said biological material to be analyzed,

said method comprising steps of:
injecting the sample via the first channel in order to concentrate the target biological species in the first space of the chamber, this being achieved via filtration,
injecting via the first channel a first solution for achieving selective-capture reaction conditions,
injecting via the first channel a solution of a hybrid first reagent comprising molecules for selectively capturing the target biological species, which are bound to an oligonucleotide sequence suitable for initiating a biomolecular-amplification reaction,
injecting via the first channel a second solution for achieving biomolecular-amplification reaction conditions,
injecting via the first channel a solution of a reagent suitable for achieving biomolecular amplification,
detecting, via biomolecular amplification, directly in the device.

According to one particularity, the method comprises a step of rinsing the target biological species concentrated in the lower space of the device, after the step of injecting the sample.

According to another particularity, the method comprises a step of rinsing the target biological species, which step is implemented after the selective-capture reaction.

According to another particularity, the step of detecting via amplification is carried out via fluorescence, colorimetry, pH-metry, turbidimetry, or lensless imaging.

According to another particularity, the first reagent comprises capturing molecules of antibody or aptamer type or of MIP type.

According to another particularity, the second reagent comprises amplification reaction initiators.

### BRIEF DESCRIPTION OF THE FIGURES

Other features and advantages will become apparent from the following detailed description, which is given with reference to the figures listed below:

FIG. 1 shows the device employed to prepare the sample and to detect the biological species.

FIG. 2 illustrates the various steps, E1 to E8, of the method of the invention, implemented in the device of FIG. 1.

FIG. 3 also illustrates the principle of preparation of the sample and of capture of the target biological species prior to their detection.

### DETAILED DESCRIPTION OF AT LEAST ONE EMBODIMENT

The invention relates to a method implemented in a device that makes it possible to concatenate preparation of a sample for a detection via selective capture, and a detection via biomolecular amplification.

The solution allows the presence of target biological species in a biological sample to be detected.

The biological sample for example takes the form of a fluid.

By biological species, what is notably meant is micro-organisms, cells, spores, viruses, toxins, etc.

By fluid, what is notably meant is a liquid, a gas, etc. The liquid will possibly have various degrees of viscosity and will for example possibly take the form of a paste or a gel.

The method notably allows target biological species present in the sample to be detected using the same device, whether via reaction of selective-capture type or via a biomolecular-amplification reaction.

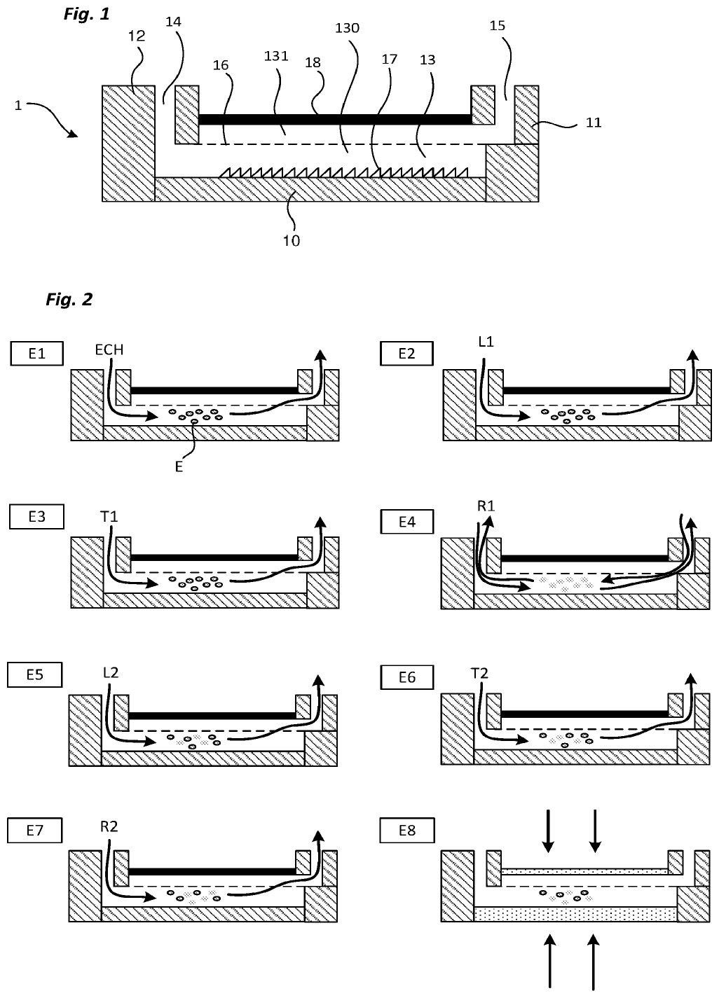

According to one particular aspect of the invention, the employed device is such as described below, with reference to FIG. 1.

The preparing device may be included in a more global detecting system, incorporating detecting means suitable for implementing a detection via amplification. Nonlimitingly, these detecting means are for example chosen from: optical hardware for detecting fluorescence, colorimetry, a pH-meter, hardware for detecting via lensless imaging, hardware for detecting via turbimetry, etc.

The device 1 comprises a casing comprising a lower wall 10, a lateral wall 11 and an upper wall 12. All of the walls of the casing will preferably be made of the same material. This material will notably be able to undergo heating in a temperature interval comprised between 20° C. and 100° C. Preferably, certain walls of the casing will be made of a transparent material. Preferably, the employed material will be a plastic, for example PMMA (polymethyl methacrylate) or COC (cyclic olefin copolymer).

The device 1 comprises a chamber 13 produced in the casing. This chamber is the location in which both the purification/concentration and the detection of the target biological species take place. The chamber 13 is closed at the bottom by the lower wall of the casing.

The device comprises a first channel 14 produced in the casing and arranged to inject fluids into the chamber or to evacuate fluids from the chamber. The first channel 14 comprises a first end comprising an aperture produced for example through the upper wall 12 of the casing and a second end that emerges into said chamber 13. The first end of the first channel 14 is for example arranged vertically and its second end emerges for example horizontally into the chamber 13. The first end of the first channel is for example flared with a view to applying thereto the cone of a pipette or will be adapted to the type of device employed to inject the fluid into the device. By way of example, it may be a question of an aperture that has an end fitting of “luer” type with a view to connecting a syringe thereto or that is suitable for the connection of a fluidic circuit such as that which will be described below.

The device comprises a second channel 15 produced in the casing. This second channel 15 also comprises a first end that communicates with the exterior, forming an aperture produced for example through the upper wall of the casing and a second end that communicates with the space formed by the chamber 13. Via this second channel 15, it is also possible to inject fluids into said chamber or to evacuate fluids from said chamber. Its first end is for example arranged vertically and its second end horizontally. The chamber 13 is placed between the first channel 14 and the second channel 15. Identically, the first end of this second channel is for example flared with a view to applying thereto the cone of a pipette or will be adapted to the type of device employed to inject the fluid into the device. By way of example, it may be a question of an aperture that has an end fitting of “luer” type with a view to connecting a syringe thereto or that is suitable for the connection of a fluidic circuit such as that which will be described below.

At the top, the chamber 13 may be closed by a membrane 18 that is advantageously supple and stretchable, and that is preferably transparent. The upper wall 12 of the casing of the device thus comprises an aperture that is covered in a hermetic manner by said membrane 18. Said membrane is thus anchored to the casing by any suitable fastening solution, by adhesive bonding for example. This membrane 18 will for example be composed of a film, for example a self-adhesive PET film, of thickness, of dimensions and of make-up that make it able to deform elastically, with respect to its anchoring points, notably as far as to the bottom of the chamber 13.

By the term “transparent”, what is meant is that the employed material is at least partially transparent to visible light, to fluorescence or to luminescence, so as to let past at least 80% of this light. It must thus be understood that it will be sufficiently transparent to see the interior of the chamber 13, and at least the second space located above the filter 16 mentioned below.

The device comprises a filter 16 arranged in said chamber 13 and separating said chamber 13 into two spaces. The two spaces are for example superposed and thus designated the lower space 130, this space being located under the filter, and the upper space 131, this space being located above the filter and under the membrane 18. All or some of this filter 16 preferably takes the form of a thin and supple film held in the space formed by the chamber so as to allow passage from one space to the other only via the pores of the filter 16. The film advantageously has an elastic deformability that allows it to stretch during the exertion of a bearing force in a substantially vertical direction, this elastic deformability having a sufficient level to reach the lower surface of the chamber 13. The filter 16 has an average pore diameter comprised between 10 nm and 50 μm, and for example comprised between 0.2 μm and 1 μm for the separation of microorganisms. The diameter of the pores is of course chosen to ensure a separation between various biological species present in the sample. The filter 16 will for example be composed of a film of thickness, of dimensions and of make-up allowing it to deform as far as to the bottom of the chamber 13 with respect to its anchoring points. According to one particular embodiment, the filter will also possibly be made of a transparent material, for example one with the same transparency characteristics as the membrane. For bacteria, the filter may have a pore diameter ranging from 0.2 to 2 μm to retain the bacteria.

If a lysis is necessary, for example to free intra-cellular viruses, the device may advantageously comprise a rough backing surface 17 arranged on the bottom of the chamber 13. This rough backing surface 17 extends over most of the bottom of the chamber. It has an average surface-roughness parameter comprised between 0.1 μm and 10 μm, and preferably comprised between 0.2 μm and 3 μm. This rough backing surface 17 is intended to allow a mechanical lysis of the biological species present in a biological sample placed in the device. Preferably, the mechanical lysis is achieved by grinding said biological species, via abrasion on said rough backing surface. The grinding operation is implemented via a frictional movement of the biological species against the rough backing surface, using a suitable grinding member. This member will for example be a spatula or a rod, for example made of plastic or metal. This member is applied from the exterior of the chamber 13 and its end is applied against the external surface of the membrane 18 so as to stretch the membrane 18 and the filter toward the bottom of the chamber and thus rub the biological species present in the sample against the rough backing surface 17.

Preferably, the casing may advantageously incorporate means for heating the internal space of the chamber, which means are for example composed of at least one resistive heater. The resistive heater is for example fastened to the lower wall of the casing. A power source will for example be provided with a view to powering the resistive heater. The power source will for example comprise one or more electrical batteries, which deliver enough power to heat the chamber to a temperature comprised in the interval defined above, i.e. from 20° C. to 100° C. Of course, other heating means could be employed, these means for example comprising a conductive ink deposited by printing press or screen-printing under the lower wall of the casing.

Thus, to summarize, the device may advantageously comprise the following “multilayer” structure:
optionally, a rough lower backing surface 17;
a lower space 130 of the chamber 13, which space is located above the rough backing surface 17;
a filter 16, which is advantageously supple and stretchable and which is located above the lower space 130;
an upper space 131 of the chamber 13, which space is located above the filter 16;
a membrane 18, which is advantageously supple and stretchable and which is located above the upper space 131, hermetically closing the chamber and accessible from the exterior of the device.

Nonlimitingly, the device may have the following dimensional characteristics:
a first channel 14 composed of an inlet channel of 1 mm diameter×3 mm height, then of a channel of rectangular 1 mm×150 μm cross section of 3 mm length;
a chamber 13 composed of a lower space 130 for concentration/lysis that has a diameter of 8 mm and a height of 150 μm, and of an upper space for elution with a diameter of 8 mm and a height of 300 μm;
a filter 16 of porosity tailored to the target to be retained (virus, yeast, fungus, etc.) for example with a porosity of 0.2 to 2 μm to retain bacteria or of 10 to 100 nm to retain viruses;
a second channel 15 composed of a channel of rectangular 1 mm×150 μm cross section of 3 mm length, then of a channel of 1 mm diameter×3 mm height.

Starting with this device, with reference to FIG. 2, the method of the invention comprises the following steps:

E1: the sample ECH (for example in liquid form) is injected into the device 1 via the first channel 14, the target biological species E are held in the lower space 130 of the chamber 13 and concentrated in this space.
E2: the target biological species E trapped in the lower space 130 of the chamber 13 are rinsed with a suitable buffer L1, which is injected via the first channel 14 and evacuated via the second channel.
E3: the target biological species E are conditioned for selective capture. A suitable buffer T1 is injected via the first channel 14 and evacuated via the second channel 15 of the device.
Steps E2 and E3 may be merged and combined into a single step.
E4: a hybrid reagent R1 suitable for the selective capture is injected via the first channel 14 of the device. This reagent may comprise capturing molecules of antibody, aptamer or MIP type (MIP standing for molecular imprinted polymer) or other known compounds. The target biological species E concentrated in the lower space 130 of the chamber 13 are thus recognized by the capturing molecules. The reagent R1 may be flowed back and forth in and out of the chamber, via the first channel 14 and the second channel 15, with a view to improving the probability of the target biological species E and the capturing molecules meeting.
E5: this is a step in which the species captured in the lower space of the chamber are rinsed by injecting a rinsing buffer L2. This step E5 allows non-specifically absorbed capturing molecules to be cleaned away. Just as in the preceding step, it is possible to flow the rinsing solution L2 back and forth through the chamber 13 of the device.
E6: this is a step of rinsing with a buffer T2, suitable for achieving the conditions of the biomolecular-amplification reaction. The buffer is injected via the first channel 14 of the device and evacuated via the second channel 15.
Steps E5 and E6 may be merged and combined into a single step.
E7: this is a step of injecting a reagent R2 suitable for the implementation of the amplification reaction.
E8: the amplification reaction occurs. Some of the amplicons may migrate through the filter 16 of the device. The amplification reaction may require the device to be heated and/or cycled. Detection may be achieved by reading the reaction via fluorescence, colorimetry, pH-metry, or lensless imaging. Depending on the method employed, amplification curves may be drawn with a view to obtaining qualitative responses (presence/absence) and quantitative responses regarding the target species. The detecting means of the system are tailored to the type of detection implemented (optical hardware for detection of fluorescence, colorimetry, a pH-meter, hardware for detection via lensless imaging, etc.).

It will be noted that the device must be tailored to the type of detection performed. It may comprise one or more transparent walls, and the filter 16 and the membrane 18. In case of detection via lensless imaging, the chamber is visible from above and from below, as illustrated by the arrows shown in step E8 of FIG. 2.

The principle of the biomolecular detection may rely on coupling of a reagent for recognizing the target injected in step E1 to an oligonucleotide sequence, which is preferably a dumbbell oligonucleotide sequence, or any other oligonucleotide sequence allowing the initiation of a LAMP reaction (LAMP standing for loop-mediated isothermal amplification) or of a biomolecular-amplification reaction. The obtained reagent thus corresponds to the reagent R1 of aforementioned hybrid type.

The recognizing molecule, which is an antibody (or any other capturing molecule), specific to the target biological species E to be detected, and employed in the selective-capture reaction, is covalently bonded to a dumbbell sequence of oligonucleotides the design of which is such that said sequence may be amplified isothermally with at least two initiators. This dumbbell is synthesized beforehand chemically and is designed so as to be as short as possible (at most 200 nucleobases) in order to optimize its manufacture and to then accelerate the amplification reaction. It may be identical and generic whatever the target, and the initiators for amplifying it are specific to this dumbbell. The time of conception of the biomolecular reaction is therefore greatly shortened with respect to a conventional amplification reaction.

In the case of use of an aptamer as recognizing reagent, the latter may be integrated directly into the dumbbell amplification sequence. In this case, the use of antibodies, which may be not very stable over time, and which are time-consuming and expensive to produce and tricky to use because of their high temperature sensitivity, is completely avoided. There is also no need to couple the dumbbell to the antibody, the steps required to do so possibly proving to be low-yield. The good affinity of the aptamer to the target makes it possible to ensure the specificity of the reaction.

Contrary to conventional biomolecular amplification techniques, this technique does not directly amplify the DNA of the target, but an oligonucleotide sequence contained in the hybrid reagent R1 allowing the target to be selectively recognized. Thus, no lysis of the sample is required.

The use of at least two initiators allows the amplification reaction to be clearly accelerated. It occurs in less than 30 minutes at a constant temperature of 65° C.

This reaction principle is notably described in the patent application filed under number FR2002366, which application was filed on Oct. 3, 2020 and is titled “Procédé pour détecter et éventuellement quantifier un analyte avec un oligonucléotide à double structure tige-boucle et ledit oligonucléotide”, which may be translated “Method for detecting and optionally quantifying an analyte with an oligonucleotide of double stem-loop structure and said oligonucleotide”.

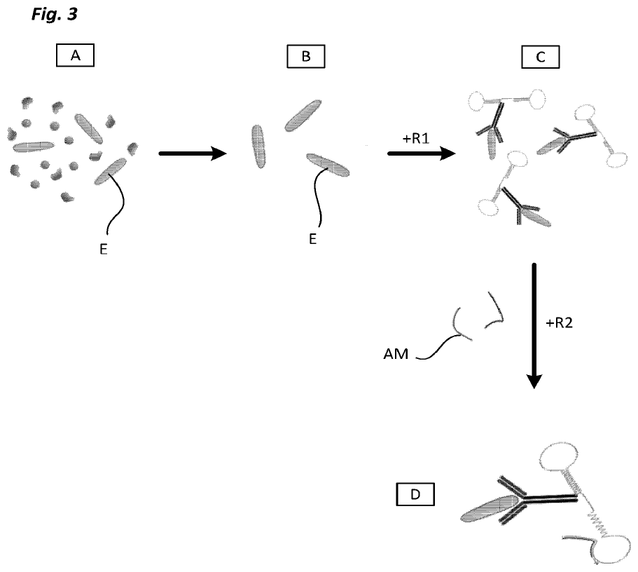

FIG. 3 illustrates the implementation of the method. FIG. 3 thus shows:

Step A: the complete sample ECH, which comprises the target biological species E and other undesired components.

Step B: after concentration in the device in step E2, the sample no longer comprises anything but the concentrated and partially purified target biological species E in the lower space 130 of the chamber 13.

Step C: it is a question of adding the hybrid, selectively capturing reagent R1, as provided for in step E4. The capturing molecules graft to the target biological species E.

Step D: it is a question of adding the reagent R2, which is formed from the initiators AM required by the biomolecular-amplification reaction, as provided for in step E7.

It will be noted that a step of lysing the biological species present in the sample ECH could also be carried out. This optional lysing step could be implemented between steps E2 and E3 described above, i.e. after the concentrating step. It would be useful in the case where the target biological species E are not directly accessible in the sample. In the case where the lysing step is necessary, the filter 16 must be adapted.

The detecting method of the invention has the advantages defined below:
Sample preparation, which is carried out in a manner common to the selective capture and the biomolecular amplification, is very simple, and sequentially adaptable to each step.
The preparation of the sample in the device is very fast (<10 minutes).
The method does not divide the sample: no fractionation required to place the sample in the right reagent, and therefore no dilution.
All of the target species present in the sample are engaged in the detecting reaction (no dilution, use of all of the eluate, etc.).
Each step is carried out directly in its reagent (no dilution or fractionation of the sample to prepare it and detect it).
This method for preparing samples enables concentration and purification and advantageously comprises no lysing step.
Sensitivity is comparable to or higher than the reference immunology or PCR methods.
Specificity differs depending on the target, the strain, the type of receiver, etc.
It is implemented using a single device, to concatenate selective capture and biomolecular detection.

1. A method for detecting target biological species in a biological sample, said method being implemented in a detecting system that comprises a preparing device and detecting means, said preparing device comprising:
a casing comprising at least one aperture,
a first channel produced in said casing,
a second channel produced in said casing,
a chamber into which emerge the first channel and the second channel,
a filter separating said chamber into two separate spaces so as to define a first space into which emerges said injection first channel and a second space into which emerges said second channel, said filter having a porosity suitable for retaining said biological material to be analyzed,
said method comprising the steps of:
injecting the sample via the first channel in order to concentrate the target biological species in the first space of the chamber, this being achieved via filtration,
injecting via the first channel a first solution for achieving selective-capture reaction conditions,
injecting via the first channel a solution of a hybrid first reagent comprising molecules for selectively capturing the target biological species, which are bound to an oligonucleotide sequence suitable for initiating a biomolecular-amplification reaction,
injecting via the first channel a second solution for achieving biomolecular-amplification reaction conditions,
injecting via the first channel a solution of a reagent suitable for achieving biomolecular amplification,
detecting, via biomolecular amplification, directly in the device.

2. The method as claimed in claim 1, said method further comprising a step of rinsing the target biological species concentrated in the lower space of the device, after the step of injecting the sample.

3. The method as claimed in claim 1, said method further comprising a step of rinsing the target biological species, wherein said step of rinsing is implemented after the selective-capture reaction.

4. The method as claimed in claim 1, wherein the step of detecting via amplification is carried out via fluorescence, colorimetry, pH-metry, turbidimetry, or lensless imaging.

5. The method as claimed in claim 1, wherein the first reagent comprises capturing molecules of antibody or aptamer type or of MIP type.

6. The method as claimed in claim 1, wherein the second reagent comprises amplification reaction initiators.
